(19) United States
(12) Patent Application Publication
(10) Pub. No.: US 2004/0074500 A1
(43) Pub. Date: Apr. 22, 2004

(54) Oxyframe

(76) Inventor: Kacey Gene DePuy, Banning, CA (US)

Correspondence Address: Kacey Gene DePuy, 1396 Wyte Way, Banning, CA, 92220, US

(21) Appl. No.: 10/642,838

(22) Filed: Aug. 18, 2003

Related U.S. Application Data
(60) Provisional application No. 60/407,675, filed on Sep. 3, 2002.

(51) Int. Cl. G02C 1/00; A62B 7/00; A61M 15/08

(52) U.S. Cl. 128/207.18; 351/83

(57) ABSTRACT

In the past with a person who wore eyeglasses and had to be on oxygen at the same time, usually that person would have to wear the arms from the eyeglasses and the airtube that carries the oxygen to their nose on their ears simultaneously. What I'm proposing is, combine eyeglass frames and the tube that carries the oxygen together. How that would be done is, making the eyeglass frames hollow, starting from the arms of the glasses, around the eye pieces, down below to the nipples that would connect to the nose piece, and finally in the nostrils. With this combination a person could wear glasses and breath with the oxygen at the same time with more ease. That makes for less adjustment, and it's much less frustrating than the old way.

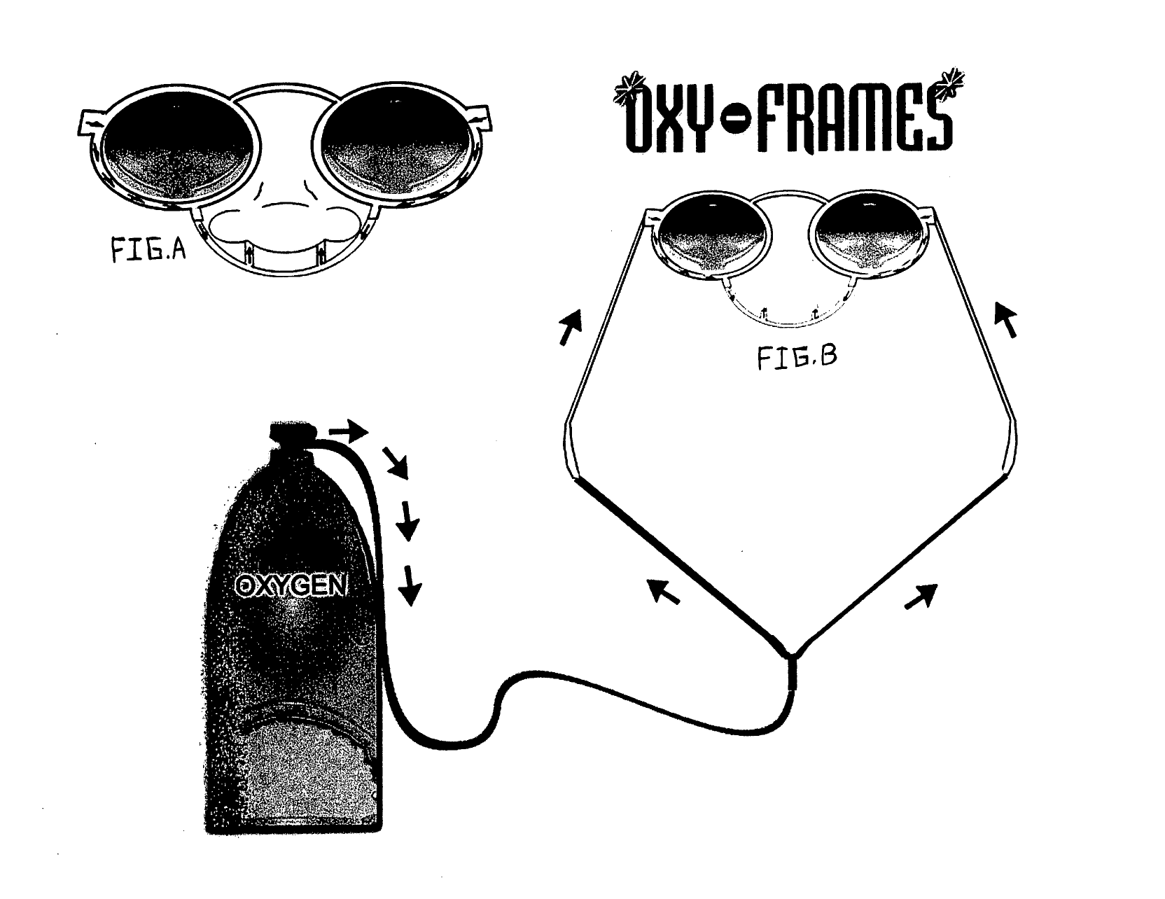

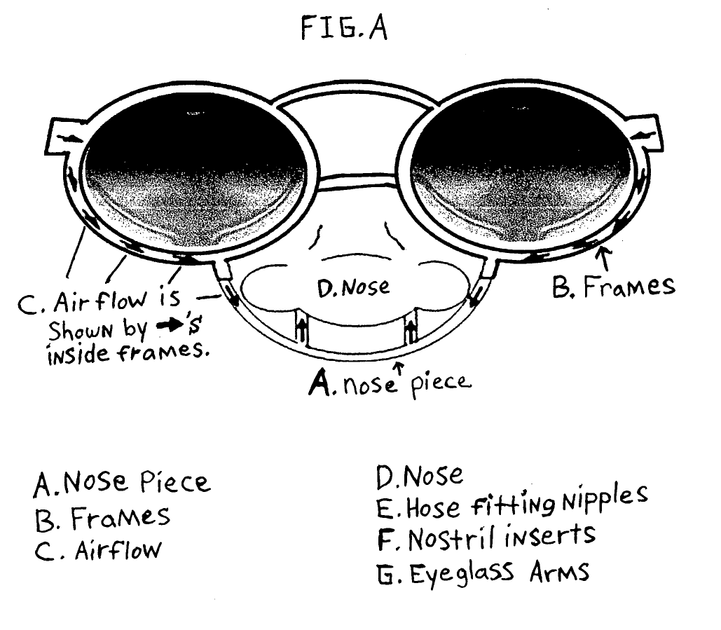

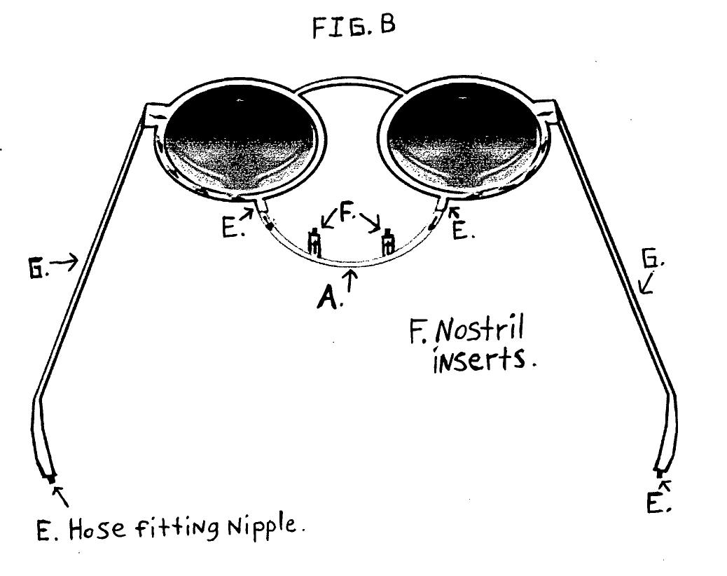

### SPECIFICATION

This idea is a combination of eyeglasses and the breathing tube that a person uses when on oxygen. What happens is the eyeglasses are hollow from the end of the arms around the lenses down to the hose fitting nipples, which reaches the nose piece, into the nose. Both arms have a single nipple that attaches to the oxygen tube that runs to the oxygen tank.

The eyeglass frames are made of common materials in the process of making frames.

1. What I claim my invention to be is the combination of eyeglass frames, and the airtube that carries oxygen from an oxygen tank to the nose. This makes the eyeglass frames hollow to allow airflow.
